(19) United States
(12) Patent Application Publication
(10) Pub. No.: US 2009/0216339 A1
(43) Pub. Date: Aug. 27, 2009

(54) Through-Liner Electrode System for Prosthetics and the Like

(76) Inventors: William J. Hanson, Bolton, MA (US); T. Walley Williams, III, Belmont, MA (US)

Correspondence Address: MIRICK, O'CONNELL, DEMALLIE & LOUGEE, LLP, 1700 WEST PARK DRIVE, WESTBOROUGH, MA, 01581, US

(21) Appl. No.: 12/348,179

(22) Filed: Jan. 2, 2009

Related U.S. Application Data
(60) Provisional application No. 61/018,525, filed on Jan. 2, 2008.

(51) Int. Cl. A61F 2/70; A61B 5/103

(52) U.S. Cl. 623/25; 600/587

(57) ABSTRACT

A system for passing myoelectric signals through a suspension liner of a prosthetic device, the suspension liner having an inner surface that is in contact with a user's skin and an outer surface that is adjacent to an outer socket of the prosthetic device. The system in one embodiment includes a flexible conductive electrode insert defining a first portion located on or adjacent the inner surface of the suspension liner such that it touches the user's skin, a second portion passing through the suspension liner, and a third portion on the outer surface of the suspension liner, and an adhesive that adheres at least the second and third portions of the insert to the suspension liner.

### CROSS REFERENCE TO RELATED APPLICATION

This application is based upon and claims the benefit of priority of U.S. provisional application No. 61/018,525, filed on Jan. 2, 2008, the entire contents of which are incorporated by reference herein.

### FIELD OF THE INVENTION

This invention relates to prosthetics and more specifically to a system for acquiring myoelectric signals through suspension liners for operating myoelectrically controlled powered prostheses, and to the general field of medicine for acquiring bioelectric signals through non-conducting barriers.

### BACKGROUND OF THE INVENTION

Myoelectric signals picked up from an amputee's residual limb are sometimes used to operate advanced upper extremity prosthetic systems. In some case, the prosthesis is suspended from the residual limb by a liner that is rolled over the amputee's residual limb. The liner is typically made of silicone. Such liners are a low-cost way to provide comfortable suspension of radial- and humeral-level prostheses for amputees using myoelectric control of their prosthesis. However, as silicone is not conductive, the liner is not able to pass the necessary myoelectric signals. Passing these electrical signals through the thickness of the liner is a problem that has plagued prosthetists for some time. The need is to transmit myoelectric signals through the thickness of an elastomeric (roll-on) suspension liner, without creating openings in the liner that may reduce or eliminate the effectiveness of suction suspension provided by these liners. This is an issue in both upper-extremity and lower-extremity prostheses.

Several through-liner electrode systems have been developed to facilitate the acquisition of myoelectric signals in a prosthesis employing suspension liners. These generally consist of a conductive material inserted into the suspension liner that passes the myoelectric signal through the non-conductive liner. This has been done on custom liners where a conductive material is molded into the liner during manufacture. This has several drawbacks, including accuracy of the conductive material placement, delays in manufacturing “custom” liners, additional labor/cost to the clinician in preparing a custom liner, and proper alignment of the conductive material with the electrode contacts in the socket.

### PRIOR ART

A prior patent describes ways to transmit myoelectric signals through an elastomeric patch. U.S. Pat. No. 5,443,525 describes an elastomeric patch to be bonded on the inside surface of a liner to transmit signals from the inside of the liner to the outside. Approximately 40,000 thin conductors allow a metal electrode on the outside of the patch to sample the signal on the underlying skin. Two or more metal electrodes can sample adjacent areas because the patch conducts only from the inside to the outside, but not laterally. The patent describes the conductive material as being bonded with its outer surface in contact with the inner surface of the liner, leaving an uncomfortable discontinuity at the outside of the patch. Thus the patent fails to provide a finished liner of approximately uniform thickness with localized compression into the amputee's skin only to the degree needed to collect a reliable myoelectric signal. Furthermore, the area sampled depends upon the area of good contact between the conducting patch and the metal electrode touching the outer surface, thus the effective contact can change as pressure is applied between the electrode and patch as might occur when lifting a heavy object.

Also known in the art is a method for making custom liners with conducting material molded into the liner and cured at the same time as the liner material. These liners may work well clinically, but are not practical for the prosthetic technician to fabricate locally. Further this method does not address the issue of providing various conductive surface areas on the inside and outside of the liner or varying degrees of localized compression of the underlying skin. Also, it does not address the issue of providing greater bonding strength between the liner and insert than is provided by a common butt joint. This may be required to prevent the insert from separating from the liner during repeated donning. If the insert is co-cured with the liner, this would not be a butt joint, but if a pre-formed insert is held in place on the cast while new silicone flows around it, it is indeed a butt joint.

### SUMMARY OF THE INVENTION

It is therefore a primary object of this invention to provide a system of conductive inserts for use in suspension liners to obtain myoelectric signals from an area of the user's skin surface which does not change as pressure over the area changes, and to transmit these signals to conductors in the socket outside the liner to supply control signals to a powered prosthesis or for other applications in which myoelectric signals need to be transmitted.

It is a further object of the invention to provide a multiplicity of prefabricated conductive inserts to suit the particular needs of the user.

It is a further object of the invention to provide a system for mounting these conductive inserts into commercial (off-the-shelf) suspension liners.

It is a further object of the invention to provide conductive inserts with high conductivity in all directions (isotropic).

It is a further object of the invention to provide conductive inserts with properties that make the inserts attractive to magnets.

It is a further object of the invention to provide conductive inserts with the right conductive characteristics to effectively pass the myoelectric signal through the suspension liner.

It is a further object of the invention to provide conductive inserts with uniform conductivity so that the contact of a conducting electrode on the outside of a conductive insert provides essentially the same signal regardless of the location or minor repositioning of this contact point.

It is a further object of the invention to provide a multiplicity of conductive inserts with suitable shapes (e.g., domes) to assure good contact with the user's skin surface and sufficient compression of the underlying tissue to adequately sample signals from the underlying muscle.

It is a further object of the invention to provide a multiplicity of conductive inserts with suitable shapes to assure adequate contact with electrodes in the outer socket, particularly to accommodate minor circumferential mis-alignment.

It is a further object of the invention to provide conductive inserts where the surface that contacts the skin is flat or convex (domed). A high-dome insert can compress soft tissue to acquire better quality myoelectric signals.

It is a further object of the invention to provide conductive inserts with elastic characteristics closely matched to the elasticity of the suspension liner.

It is a further object of the invention to provide a method for placing these conductive inserts in a sheet for use in applications where a suspension liner is unsuitable, for instance a sheet held against the chest wall.

It is a further object of the invention to provide a multiplicity of conductive inserts for use with different thickness liners.

It is a further object of the invention to provide a system where the clinician can tailor the spacing and location of the conductive inserts to obtain the optimal myoelectric signal from the user's muscles.

It is a further object of the invention to provide a conductive insert with a shape and structure that assist with a good bond with the liner through the use of a relatively large surface area in shear rather than depending on bonding of the butt joint alone.

It is a further object of the invention to provide a conductive insert with optional fabric embedded in the conductive material for additional reinforcement and greater bonding strength.

It is a further object of the invention to provide a means to increase the contact area of the outer surface of the conductive inserts and to accommodate some misalignment of the liner and socket, both axially and circumferentially.

It is a further object of the invention to provide non-conductive inserts with similar physical characteristics to the conductive inserts together with an appropriate adhesive, to allow a clinician to repair errors in the placement of inserts in the suspension liners.

It is a further object of the invention to provide conductive inserts made in single or multiple-cavity molds such that the exact diameter of the portion passing through the suspension liner can be controlled along with the thickness and the shape of the surface that will contact the user's skin.

It is a further object of the invention to provide conductive inserts wherein the conducting material is attracted to a magnet.

It is a further object of the invention to provide magnetic electrodes which are attracted to conductive inserts with magnet attracting properties and therefore to provide good conductivity as well as to assure proper contact and alignment when an outer socket with electrodes is not present or when it is more convenient to apply electrodes on the ends of cables directly to the inserts.

It is a further object of the invention to provide magnetic electrodes applied to the outside of the suspension liner where metal electrodes pierce the liner (as an alternative to the conductive inserts described above) to capture myoelectric signals for prosthetic control.

It is a further object of the invention to provide conductive inserts where the surfaces to contact the user and to contact an electrode on the outside are protected by a removable film during installation to prevent contamination by the adhesive.

It is a further object of the invention to provide conductive inserts comprising conductive fabric in place of conductive silicone or other elastomer.

This invention features a system for passing myoelectric signals through a suspension liner of a prosthetic device, the suspension liner having an inner surface that is in contact with a user's skin, and an outer surface that is adjacent to an inner or outer socket of the prosthetic device, the system comprising a flexible conductive electrode insert defining a first portion located on or adjacent the inner surface of the suspension liner such that it touches the user's skin, a second portion passing through the suspension liner, and a third portion on the outer surface of the suspension liner, and an adhesive that adheres at least the second and third portions of the insert to the suspension liner.

The first portion of the insert may define a projection that projects past the suspension liner's inner surface. The projection may be dome shaped. The third portion may have a larger surface area than does the first portion. The third portion may be a thin, flat structure, the second portion may be a post that projects from the third portion, and the first portion may be the distal end of the post. The post may be round, and the third portion may be generally rectangular.

The first, second and third portions may each be thin, flat structures. The insert may comprise a conductive fabric. The side of the fabric that is adjacent to the liner may carry material that is essentially impervious to the adhesive. The second portion of the insert may be essentially fully wetted with material that is essentially impervious to air, to make the portion air-impervious. The insert may be unitary. The insert may be fabricated from a conductive elastomer. The insert may be molded from conductive elastomer.

The system may further include a removable magnetic electrode that is magnetically coupled to the third portion of the insert, to allow varied positioning of the liner relative to the socket. The magnetic electrode carries myoelectric signals from the insert to the socket.

The invention also features a system for passing myoelectric signals through a suspension liner of a prosthetic device, the suspension liner having an inner surface that is in contact with a user's skin, and an outer surface that is adjacent to an inner or outer socket of the prosthetic device, the system comprising a multi-part electrode passing through the liner, the electrode comprising an inner portion located on the inner face of the liner and that presents a top that touches the user's skin, an outer portion located on the outer face of the liner, and an intermediate portion that electrically and mechanically interconnects the inner and outer portions, wherein the faces of the inner and outer portions that are in contact with the liner are grooved, to increase the contact area between these faces and the liner, as well as to variably compress the liner material, both of which provide a tighter grip between the electrode and the liner.

The intermediate portion may be a threaded stud that is received in a threaded bore in the outer portion. The system may further include a removable magnetic electrode that is magnetically coupled to the outer portion, to allow varied positioning of the liner relative to the socket. The system may further include a thin pickup electrode in the socket and in mechanical and electrical contact with the outer portion of the multi-part electrode, the thin pickup electrode comprising a thin inner disc that is thin enough to be easily conformed by the technician to the curvature of the socket.

Also featured is a system for passing myoelectric signals through a suspension liner of a prosthetic device, the suspension liner having an inner surface that is in contact with a user's skin, and an outer surface that is adjacent to an inner or outer socket of the prosthetic device, the system comprising a flexible conductive fabric-based electrode insert defining a first portion located on or adjacent the inner surface of the suspension liner such that it touches the user's skin, a second portion passing through the suspension liner, and a third portion on the outer surface of the suspension liner, wherein all three portions are unitary, the first portion defines a projection that projects inwardly to slightly compress the user's skin, the third portion has a greater area than the first portion, and the second portion is essentially air-impervious, and an adhesive that adheres the first, second and third portions of the insert to the suspension liner. The system may further include a film that is essentially impervious to the adhesive, located on the surface of the insert between the conductive fabric and the adhesive, to inhibit adhesive infiltration into the conductive fabric.

### BRIEF DESCRIPTION OF THE DRAWINGS

Other objects, features and advantages will occur to those skilled in the art from the following description of the preferred embodiments and the accompanying drawings, in which:

FIG. 1 is a perspective view of an embodiment of a conductive insert for the invention with a minimal overlap.

FIG. 2 is a perspective view of another embodiment of a conductive insert for the invention with a domed convex inner surface.

FIG. 3 is a perspective view of another embodiment of a conductive insert for the invention with larger overlap.

FIG. 4 shows a prosthetic liner of the invention with two pairs of active inventive conductive inserts and a reference conductive insert.

FIG. 5 shows another embodiment of a conductive insert for the invention with larger overlap and an offset.

FIG. 6 shows a mold for making the conductive insert of FIG. 3, but with a domed top.

FIG. 7 is a cross-sectional view of the mold of FIG. 6.

FIG. 8 shows an alternative conductive insert with an extended overlap layer.

FIG. 9 shows another alternative conductive insert, with a shielded extended overlap layer.

FIG. 10 shows the use of pressure-sensitive tape or other film to protect surfaces of an insert.

FIG. 11 is a cross-sectional view of the insert arrangement of FIG. 10.

FIG. 12 is a perspective view of an alternative insert for the invention made of conducting fabric.

FIG. 13 shows a cross section of the insert of FIG. 12 after fabrication, in its use position.

FIG. 14 is a longitudinal cross-sectional view through a liner and outer socket, showing the insert of FIGS. 12 and 13 in use.

FIG. 15 is an enlarged view of a portion of FIG. 14.

FIG. 16 is a partial radial cross-sectional view of the liner and socket arrangement of FIGS. 14 and 15.

FIG. 17 is a view similar to that of FIG. 15, but showing another alternative inventive insert or electrode in use with the liner, and showing another inner socket electrode design.

FIG. 18 is a cross-sectional view of the head of the liner electrode shown in FIG. 17.

FIG. 19 is a longitudinal cross-sectional view through a liner and inner socket for the arrangement shown in FIGS. 17 and 18.

FIG. 20 is a perspective view of a magnetic electrode of the invention.

FIG. 21 is a cross-sectional view of the magnetic electrode of FIG. 20.

FIG. 22 is a sectional view of one use of the magnetic electrodes of FIGS. 20 and 21 (without the connecting cable), magnetically coupled to a conductive and magnetically attractive insert in a sleeve.

FIG. 23 is a sectional view of a magnetic electrode used with a conductive insert.

### DETAILED DESCRIPTION OF THE PREFERRED EMBODIMENTS AND METHODS

One embodiment of this invention comprises a multiplicity of prefabricated elastomeric conductive inserts that are preferably molded from a conductive silicone composition (e.g., made conductive through the additional of metal particles) or equivalent, that are installed into commercial (off-the-shelf) elastomeric roll-on suspension liners (or other types of patient interface materials) as are used in orthotic and prosthetic applications. The conductive insert materials have elastic characteristics similar to that of the suspension liner and are installed into holes in the liner using elastomeric room-temperature-vulcanizing (RTV) adhesive or a similar adhesive, suitable to bond to both the liner and the conductive inserts. In one embodiment, the holes are round and are punched with punches like those used for leather and similar substances. The exact diameter of these holes will usually be equal to or slightly smaller than the diameter of the conductive insert so that there is good contact when the insert is glued in place. Several diameters can be made available to accommodate various limb/muscle sizes, the desired sensitivity and the condition of the underlying muscles.

The use of the conductive inserts described is not limited to installation in a liner. When acquiring myoelectric or EKG signals from the trunk or elsewhere on the body, the conductive inserts may be installed in a flat sheet or a form-fitting shape held against the body by the outer structures of the prosthesis. In similar manner the non-conducting silicone or other elastomeric material can be part of a tight-fitting garment with the conductive inserts being used to permit signal acquisition on the outside of the garment.

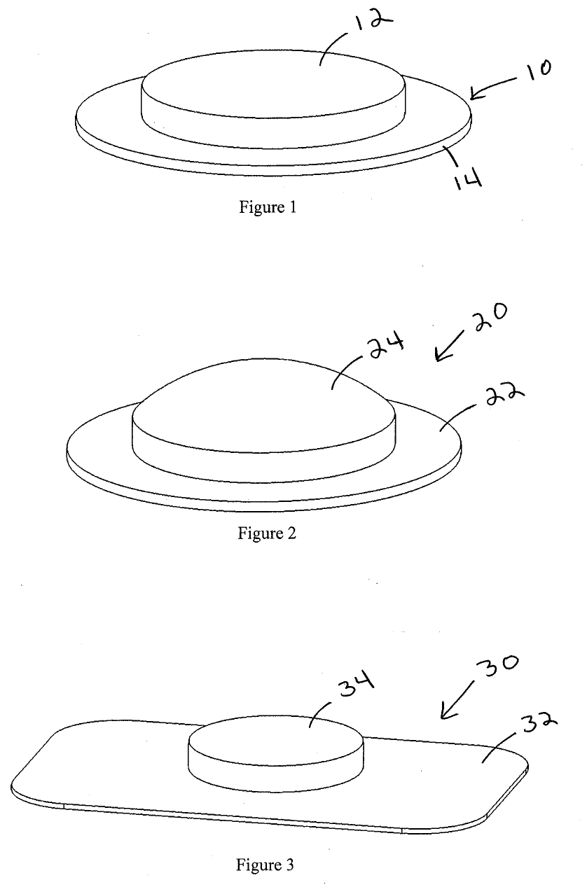

A pre-molded conductive insert 10, FIG. 1, typically has a cylindrical projecting element 12 that is approximately equal in height to the thickness of the liner into which it will be installed, typically 2-6 mm. This length may be slightly greater than the suspension liner thickness, but not less, so that the end of the projection will contact the underlying skin. Merely installing cylindrical conductive inserts with RTV silicone adhesive may not work well, because the resulting butt seam can have insufficient strength to resist tearing. This problem can be alleviated by having the portion 14 of the conductive insert outside the suspension liner larger than the cylindrical portion passing through the liner. This exterior element can be quite thin and still provide a joint that is in shear rather than in tension. This thin portion with larger area is termed herein the overlap. It may be reinforced with an open mesh fabric that is saturated with the conductive material. In its simplest form the overlap 14 is about 0.5 mm thick and 3 mm greater in diameter than the penetrating cylinder.

For convenience, the suspension liner holes and the portion of the conductive inserts to be glued to the insides of the holes are round, but it is understood that the other shapes may be appropriate for special clinical situations. For instance, square or rectangular holes and identically shaped conductive inserts permit more skin surface to be sampled while keeping the distance between conductive inserts relatively close.

In the preferred embodiment, the cylindrical projecting portion 12 of the conductive insert passing through the suspension liner is typically 2-3 mm long and 9-12.5 mm in diameter. Outside this cylindrical pass-through portion is the overlap 14, a thin conductive membrane (typically 0.5-1.0 mm thick) that is integral with the cylindrical portion and molded simultaneously. This membrane may be reinforced with an open mesh fabric that provides shear strength while still permitting passage of the signal through the openings in the mesh, or with a conductive fabric, for example.

Getting a signal through a liner is only the first step in collecting a usable myoelectric signal. Typically, a prosthesis without a liner has three metal electrodes in direct contact with the user's skin to pick up the signal from a single muscle. The two active electrodes are typically 9-12.5 mm (⅜-½ inch) in diameter and are spaced with an edge-to-edge gap of about 10-12 mm in the long direction of the limb, with a third (reference) electrode located equidistant from the two active electrodes and off to one side. One reference electrode can serve more than one pair of active electrodes. The exact location of the reference electrode is not important. These distances may be increased for larger muscles. When inventive conductive inserts are placed in a liner, they will only work if metal electrodes in the outer socket line up with (and contact) the portion of the conductive inserts (the overlap and the base of the projection) that is on the outside of the liner, adjacent the outer socket. These outer-socket electrodes pass the signals through (preferably) shielded wires to a preamplifier. Thus the spacing of the conductive inserts in the liner, and the spacing of the metal electrodes in the outer socket, should ideally be the same. Further, the orientation of the liner must be such that the metal electrodes contact the outer surface of the conductive inserts. With simple circular inserts as shown in FIG. 1 the target for the outer contact needs to be relatively small due to the close spacing necessary between projecting portions of adjacent conductive inserts.

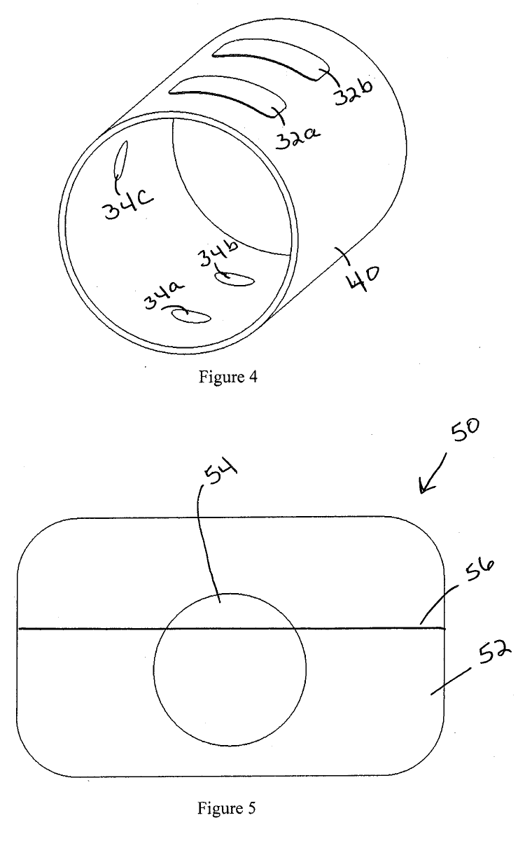

One preferred conductive insert of this invention 30 maximizes the probability of good contact by providing a large rectangular overlap 32 with rounded corners as shown in FIG. 3. This overlap accommodates greater circumferential and axial misalignment of the suspension liner and the outer socket. For instance, if the projecting portion 34 of the insert is 12 mm and the overlap is 32 mm (1¼ inch) wide in the circumferential direction of the liner, the user can miss the center of the metal electrode by 16 mm when the liner is pushed into the socket and still have adequate contact. The width of the overlap in the axial direction of the liner must be less to keep the overlaps from touching and short circuiting the signals. When the cylindrical portions of the two active conductive inserts (e.g., portions 34a and 34b, FIG. 4) are spaced 10 mm edge to edge inside the liner, a 3 mm overlap will leave a gap of 4 mm between edges of the two adjacent overlaps. This suggests that the ideal final dimensions of the outside of the conductive insert (the overlap) should be approximately 18×32 mm. FIG. 4 shows two pairs of inserts (overlaps 32a and 32b of one pair shown, and the distal ends of projecting portions 34a and 34b shown of the other pair), along with the distal end of the projection 34c from an insert for a reference electrode.

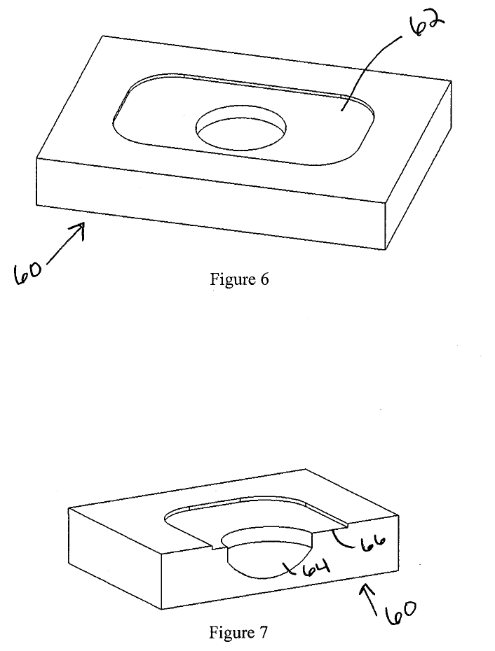

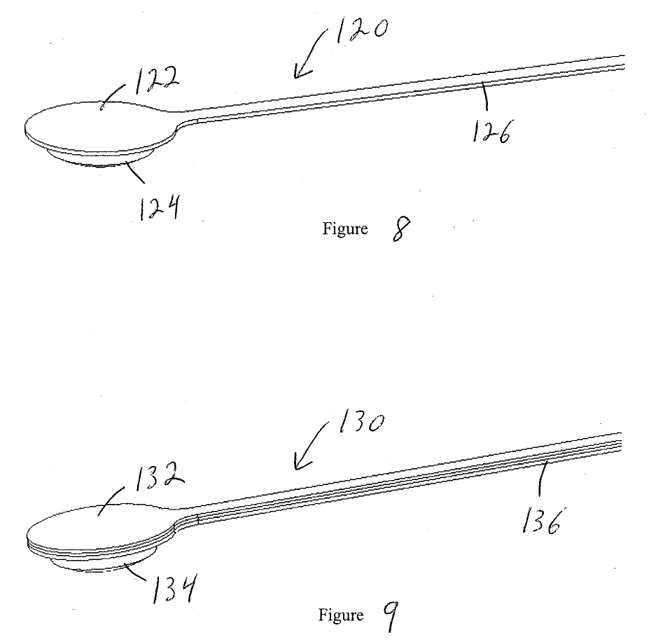

The conductive insert is preferably molded from a suitable metal-filled elastomeric material. An exemplary single-cavity mold 60 with mold cavity 62 is shown in FIGS. 6 and 7, although typically a multi-cavity mold would be used. Mold cavity 62 defines a shallow region 66 that forms the overlap, and a deeper generally semi-spherical cavity 64 that forms the domed projecting portion.

More axial misalignment can be accommodated if the pass-through portion of the conductive insert is not centered on the overlap in the axial direction. See FIG. 5, which shows insert 50 with cylindrical portion 54 offset from the longitudinal centerline 56 of overlap 52. By adding axial length on the side away from the 10 mm edge-to-edge constraint, the axial side can be increased without changing the spacing of the areas in contact with the skin. For every 1 mm of extra axial length on the overlaps of the conductive inserts, the centers of the electrodes in the outer socket can be 1 mm further misaligned. The smaller allowance for misalignment in the long direction is acceptable because roll-on liners rarely stretch significantly in this direction. This stretch may be further limited by the customary practice of having the outer surface of the suspension liner covered by a fabric engineered to control lengthwise stretch.

During a trial fitting, the conductive inserts can be positioned without actually bonding them to the liner. Also, if a location of an installed insert needs to be changed, a non-conducting insert can be bonded in place of a removed conductive insert, to fill the void. This non-conducting insert will typically have a cylindrical projecting portion the same size as that of a conductive insert and possibly a smaller overlap just sufficient to prevent tearing, because the new conductive insert location will often be close to or even overlapping the hole made by mistake.

The conductivity of the conductive inserts is important. When good low-resistance test probes are placed on both sides of a typical conductive insert, the resistance should be 4 ohms or less. This value is appropriate for the preamplifiers typically in use in prosthetics, but other values might be appropriate for amplifiers with different input impedances. Likewise, the diameter of the conductive projecting portion of the conductive insert may vary. With large limbs and muscles, larger conductive inserts with a larger diameter may be used, while with small pediatric limbs the optimal diameter may be smaller.

When there is significant fatty tissue between the muscle and the skin, it helps to have an electrode that compresses the skin's surface several millimeters. Thus the conductive inserts may be offered with flat interior surfaces as shown in FIG. 1 for some users. An alternative insert 20, FIG. 2, has overlap 22 and a protruding (e.g., convex) interior surface 24 that can sufficiently compress the skin to assist with signal pickup. When metal electrodes are mounted in sockets without liners, compression depths of 3 and 5 mm have been shown to be appropriate, and the same is true when the pickup is a conductive insert placed in a liner.

There are a number of metal electrodes that are available for mounting in the supporting socket that surrounds the liner. For use with this invention, a small diameter electrode with a minimal dome will make good contact with the conductive insert; however, contact can become intermittent if the user loses weight or muscle mass, which leaves a gap between the liner and the socket. Traditionally this problem is accommodated by deforming the supporting socket so that it comes closer to the user in the critical contact area. This same technique can be used with liners with the inventive conductive inserts.

While the primary purpose of the feed-through inserts is to install them in a roll-on liner, they can also pass myoelectric signals through permanently installed liners and then laterally. At present signals pass through such liners via a threaded post in the center of a metal electrode. The connection on the other side of the liner requires one or more nuts plus a termination on the connecting cable. The cable connection causes a bump in the outer socket to accommodate the stem of the metal electrode. A variant on the inserts of this invention can eliminate these metal electrodes and the bumps that they cause. FIG. 8 shows insert 120 with dome 124 and overlap 122. The overlap region can be extended further away from the insert by overlap extension 126; this allows the insert to reach to a convenient point for making an electrical connection. Typically there is extra space distal to the amputee stump and the inner socket for a small connection block. Such a block could easily accommodate the extensions of four active electrodes and two reference electrodes. In a typical installation, this block would also incorporate two preamplifiers. The combined block does not require any more space than is now used for two preamplifiers and their connectors.

FIG. 9 shows yet another variant of insert 130 with an extended-overlap feed-through insert. Here, the signal-conducting elastomeric layer of extension 136 is covered by an insulating layer and a second conducting layer. This second conducting layer serves as a shield against electrical noise. Two more layers may be added to the extension below the signal-conducting layer for additional shielding.

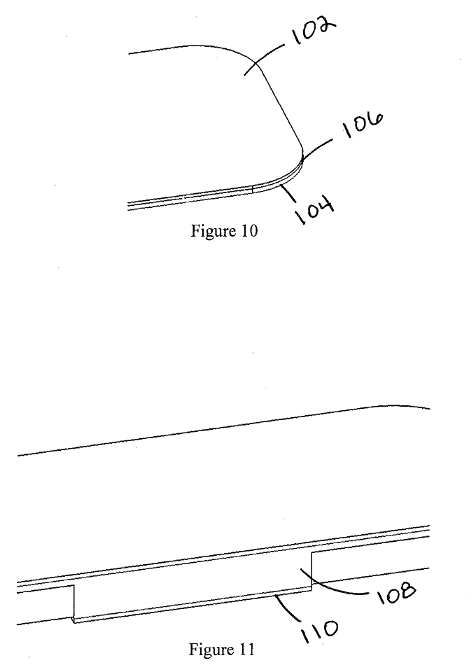

The conductive inserts can be provided in a kit to the local technician with a pressure-sensitive tape or other removable film covering the inner and outer surfaces. These covers are useful to prevent the (non-conducting) RTV adhesive from contaminating the conductive surfaces when installing the inserts in the sleeve, and to prevent other damage or contamination before installation. For example, removable films 102 and 104 can cover overlap 106, and film 110 can cover the end of projection 108, as shown in FIGS. 10 and 11.

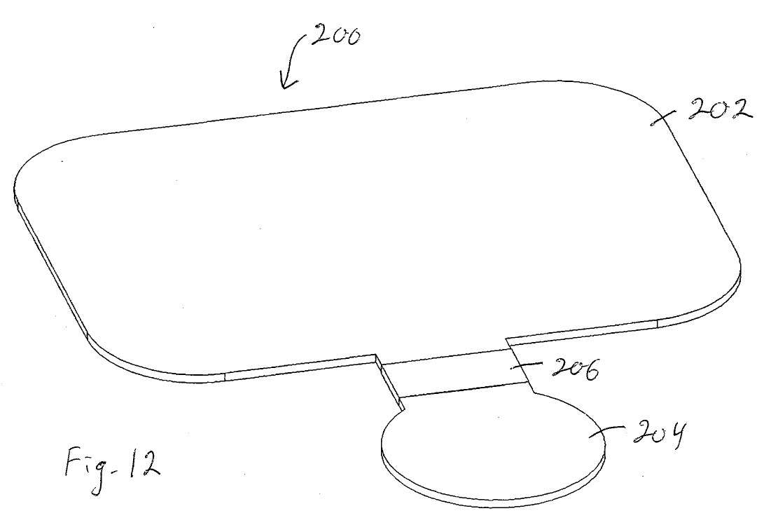

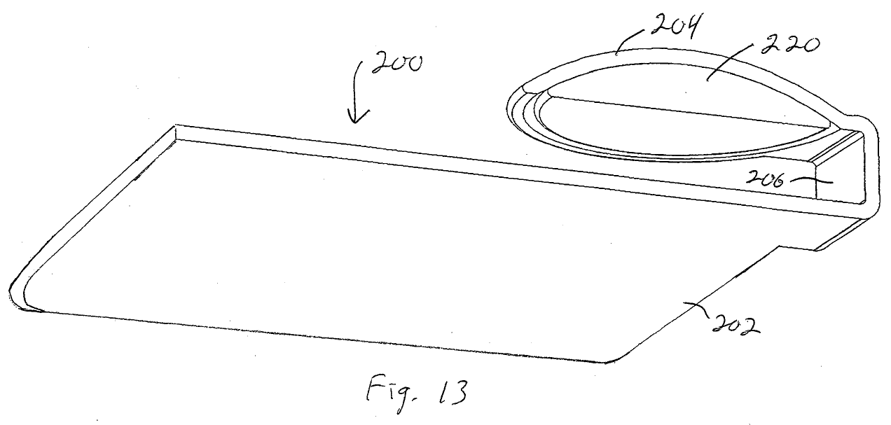

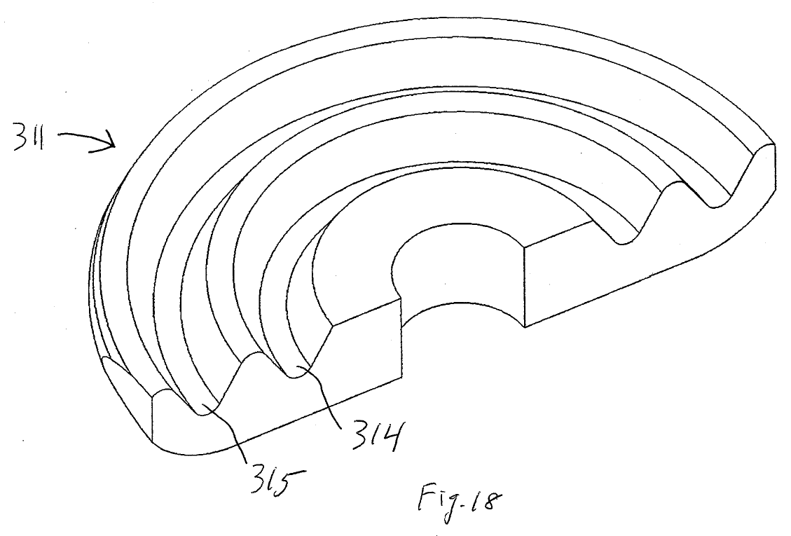

The conductive inserts can alternatively be fabricated from a conducting fabric. An embodiment of such a conductive insert 200, which has the same ability to conduct the myoelectric signal from the inside to the outside of the liner and to be easily installed by a prosthetist in a local laboratory, is shown in FIGS. 12-16 of the drawings. Insert 200 includes pickup disc 204 that is about 10 mm in diameter and outer generally rectangular overlap area 202 that is about 30 mm long.

Conductive insert 200 is made from a conducting fabric. The preferred fabric is made with a combination of coated and uncoated strands. Typically the coated strands are a made of nylon or a similar material of uniform cross section which has been coated with a thin deposit of silver. There are coated fibers running in both directions of the weave so that conducting fibers cross and therefore render the entire fabric conducting. These conducting fibers guarantee that the entire piece of fabric will act like a single conductor. In an alternative embodiment the conducting fibers are stainless steel. However, silver-coated fibers are preferred, because silver inhibits bacterial growth and the production of odors.

The conductive insert needs to be adhered to various substrates like the silicone or urethanes used for typical liners. An appropriate adhesive such as RTV silicone for silicone liners and a moisture-activated urethane for urethane liners is used to adhere the insert to the liner. It is important that the adhesive not penetrate through the fabric and create a nonconductive barrier between the insert and the skin and/or the electrode of the prosthesis. This can be accomplished by adding a layer or coating to the side of the conducting fabric that is adhered to the liner. The preferred adhesive-proof layer is a thin membrane that melts at a temperature lower than the fabric. The membrane is the same material as that used in iron-on fabric repair kits. Appropriate application of heat melts the membrane just enough to attach it to one side of the fabric.

Preferably, the insert is arranged such that no air can travel longitudinally through the portion of the fabric that passes through the liner, to prevent air from passing through the liner 230 where the insert penetrates the liner; air infiltration could have an effect on the vacuum seal between the liner and skin. An air-tight strip can be accomplished by placing a low melting-point membrane on both sides of the fabric at the correct location, and applying heat and pressure so that the melted plastic membrane saturates the fabric in this area. Alternatively, the adhesive-proof membrane can cover the entirety of one side, while an added strip of membrane is liquid enough when heated to wet through the fabric and stick to the adhesive-proof membrane on the other side of the fabric, creating a seal. Simply saturating the area with urethane adhesive will also work.

When an insert is installed in a liner, some compression of the underlying tissue by the conducting area is desirable. This can be accomplished with a dome-shaped inner insert area, as opposed to a flat area. The dome shape can be accomplished in a fabric insert by placing a shallow flexible dome under the conducting fabric area that will be inside the liner, and adhering the dome to the fabric, preferably using a meltable adhesive. For a good installation, the fibers in the fabric must slide over each other a small amount. This in turn requires the adhesive layer to melt fully while the fabric is held in contact with the dome. Ideally, the adhesive-proof layer described above will both adhere to the underside of the fabric and to the convex side of the dome. A typical dome height is about one-fourth of the diameter of the dome.

Installation of the insert requires a slit in the liner. The ends of such a slit will tend to tear even if the fabric passing through the slit is glued correctly. A small ribbon of fabric can be glued to one or both sides of the liner directly over the slit to cancel out the stress concentration at the ends of the slit.

### Fabrication of the Conductive Fabric Insert

There are several steps in the fabrication of a typical insert 200. To facilitate understanding, a 10 mm diameter pickup 204 and a 30 mm long overlap (outer conductor) 202 will be described joined by a strip 206 six by six mm to prevent the passage of air.
1. Preparation of a Strip of Conducting Fabric.
   a. A strip of conducting fabric about 50 mm wide is cut. This strip is long enough to produce a multiplicity of inserts.
   b. One side of the fabric is coated with a liquid-proof membrane.
   c. A 6 mm-wide strip of adhesive membrane is placed lengthwise in the appropriate location of the opposite side of the strip. Heat and pressure is applied to cause this strip to melt and wet through the fabric and where it adheres to the membrane on the opposite side to create an airtight seal strip 206.
2. Application of flexible domes. A row of flexible domes 220 is placed in very shallow depressions in a platen. Domes 220 are made of low durometer urethane rubber and are about 8 mm in diameter and about 1.3 mm high. The prepared strip of fabric is then placed over the domes, and a plate with a row of depressions matching the curvature of the tops of the domes is placed on top. Heat is applied to cause the domes to adhere to both the fabric and the surface of the domes.
3. Cutting of Inserts. The inserts are cut from the fabric with a laser. The laser fuses the edges during cutting and this inhibits unraveling of the fabric's fibers.

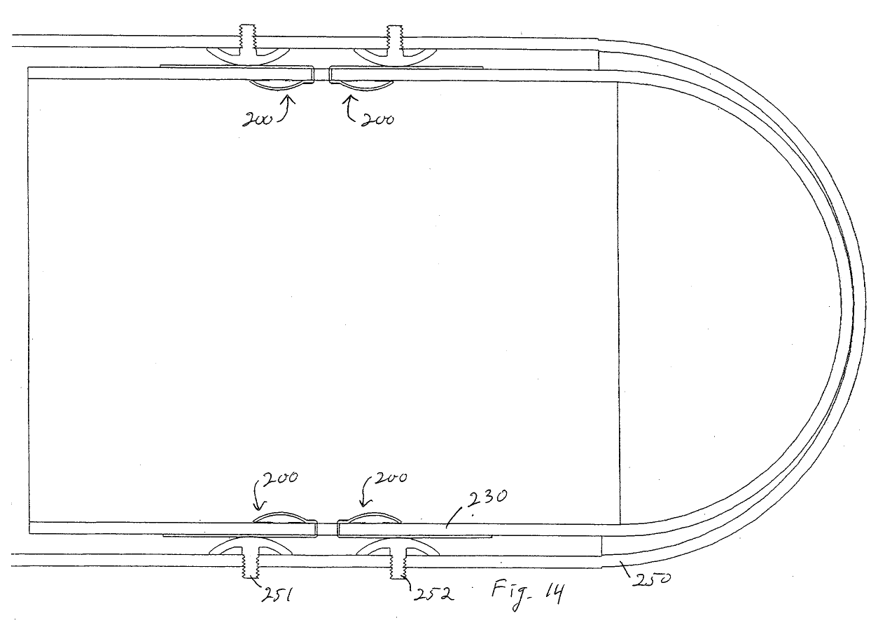

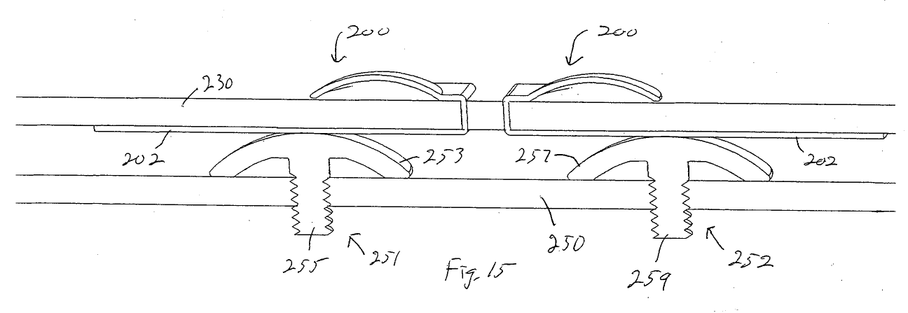

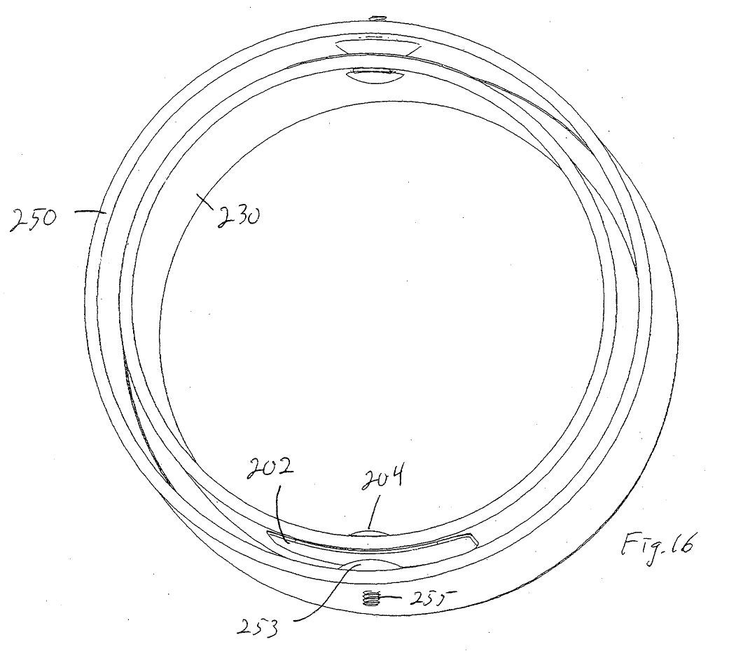

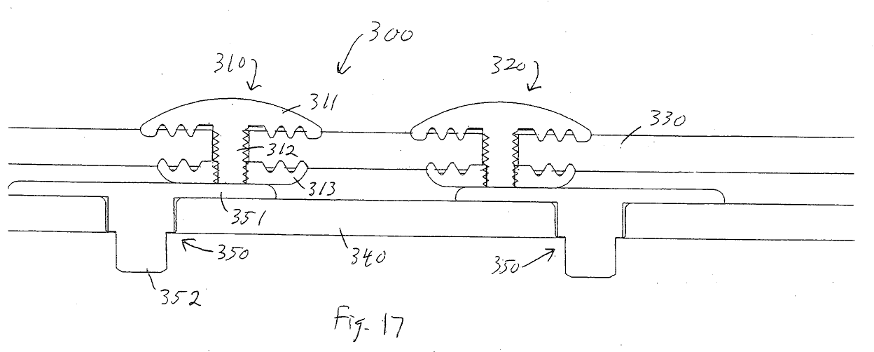

FIGS. 14-16 illustrate one of myriad potential applications of inserts 200, used on roll-on liner 230 located just inside of inner socket 250. Identical electrodes 253 and 257, with projecting studs 255 and 259, respectively, contact overlaps 202, to conduct the myoelectric signals picked up by inserts 200 through inner socket 250, to wiring that leads to electronics located in an outer socket (not shown).

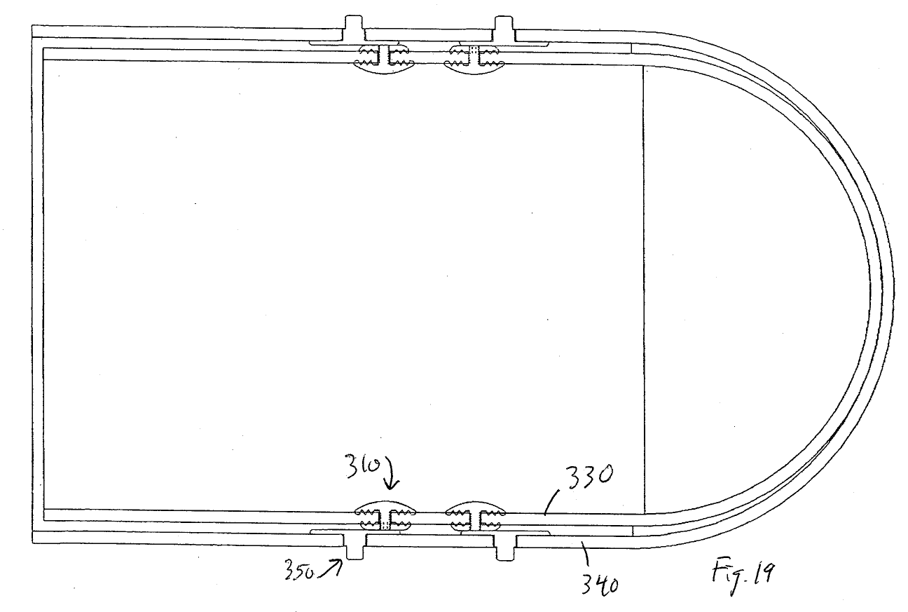

FIGS. 17-19 show another electrode design and arrangement according to the invention. Identical electrodes (inserts) 310 and 320 include an inner portion 311 that presents a domed top that will be in contact with the user's skin. Stud 312 is welded to this cap 311. Rear electrode portion 313 is threaded to receive stud 312. As best shown in FIG. 18, the inner faces of both of portions 311 and 313 are grooved (grooves 314 and 315 of portion 311 shown in the drawing), to increase the contact area between these faces and liner 330, as well as to variably compress the liner material, both of which provide a tighter grip between the insert and the liner. This arrangement helps to prevent a common problem with through-liner electrodes, which is that as the liner is stretched when it is donned or used, gaps can develop between the liner and the electrode due to the fact that the liner stretches and the electrode does not. Also, the low profile inner nut 313 prevents the lumps that are created by the use of the typical wiring attached to the outside of the liner electrodes.

Inner socket 340 carries thin pickup electrodes 350 that define a low profile as well. Inner disc 351 is thin enough to be easily conformed by the technician to the curvature of the inner socket 340. Outer shank 352 is threaded so that it can receive a nut to attach the wiring. Electrodes 350 are preferably made from annealed type 304 stainless steel, which is sufficiently malleable. For use with magnetic electrodes described below (rather than electrodes 350), the material of at least the inner nut 313 needs to be magnetic, such as a 400 series stainless steel.

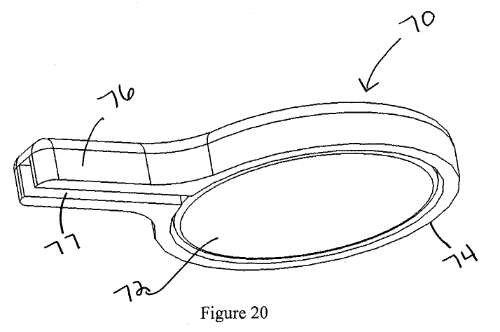

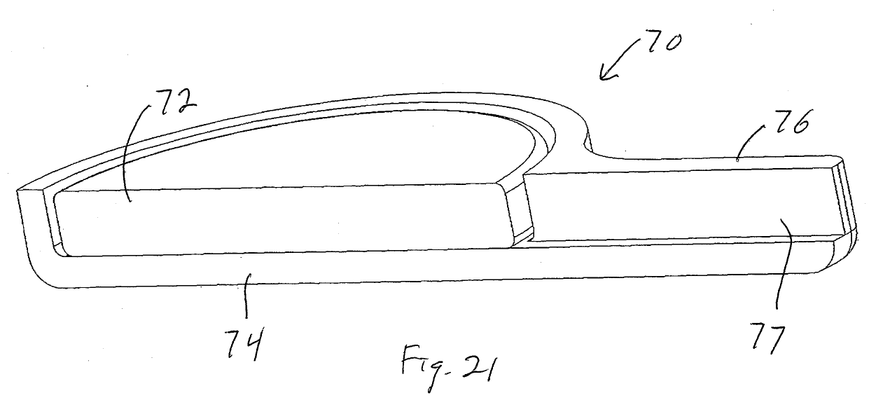

In lieu of the metal inner socket electrodes described above, an alternative is to use magnetic electrodes with attached cables, which are magnetically attracted to the conductive insert rather than being held in place by the socket. Examples are shown in FIG. 20-23.

In this case, the conductive insert contains or is made from a material that is both conductive and attractive to a magnet, e.g., containing ferrous powder (such as steel), or with two materials, one for conductivity and one for magnetic properties. Magnetic attraction assures good electrical contact and therefore good signal transmission from the conductive insert to the cable leading to the pre-amplifier. It can also help to accommodate weight loss or other changes in socket shape. Magnetic electrodes are particularly applicable when the fitter is evaluating the user prior to the fabrication of the hard socket.

Magnetic electrodes can also be used where it is desirable to leave the portion of the liner with the electrodes open to the outside of the prosthesis. In this case the magnetic electrodes can be on short cables, and placed over the conductive inserts by the user after the prosthesis has been donned.

Magnetic electrodes can also be used with more traditional sockets where a metal electrode pierces the inner socket or liner, in which case the magnetic electrode can be attached to the outside of the metal electrode, thus avoiding snaps or other similar attachment methods. In such instances the metal electrode requires an outer element that is made of a material to which a magnet is attracted, such as a 400 series stainless steel.

The magnetic electrodes 70 of this invention typically include a small high-strength magnet, in one embodiment being about 0.25 inches in diameter and about 0.03 inches thick. As shown in FIGS. 20 and 21, the magnet 72 is preferably located in a cup 74 of high permeability alloy. The cup concentrates the field on one side of the assembly and also prevents stray fields from interfering with objects in the vicinity. As shown in FIG. 21, the edge of cup 74 preferably projects slightly beyond the edge of magnet 72 to accomplish good electrical contact to the conductive insert. Protrusion 76 includes a groove 77, to allow a crimped electrical joint with the cable (not shown) that leads to the pre-amp.

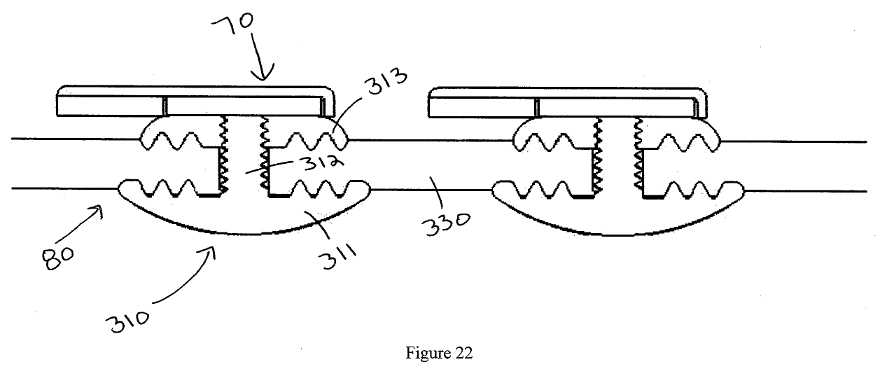

FIG. 22 shows one use of a magnetic electrode 70 in assembly 80. Grooved-back domed stainless steel electrode 310 is in contact with the user's skin (not shown), and projects through sleeve 330. Magnetic stainless steel grooved-back nut 313 fits over the distal end of stud 312 projecting from the back of electrode face 311.

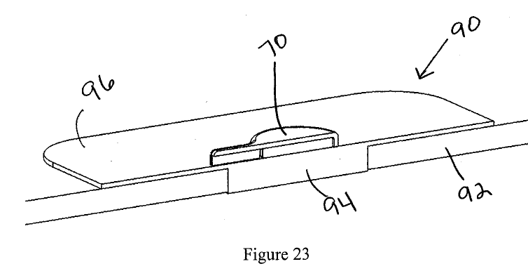

FIG. 23 is a section through magnetic electrode 70 (without the connecting cable) magnetically and electrically coupled to overlap 96 of conductive and magnetically attractive insert 90 that is located in sleeve 92. FIG. 23 also shows insert projection 94 passing through sleeve 92.

Although specific features of the invention are shown in some drawings and not others, this is for convenience only, and the features may be combined in other manners in accordance with the invention.

Other embodiments will occur to those skilled in the art and are within the following claims.

What is claimed is:

1. A system for passing myoelectric signals through a suspension liner of a prosthetic device, the suspension liner having an inner surface that is in contact with a user's skin, and an outer surface that is adjacent to an inner or outer socket of the prosthetic device, the system comprising:
a flexible conductive electrode insert defining a first portion located on or adjacent the inner surface of the suspension liner such that it touches the user's skin, a second portion passing through the suspension liner, and a third portion on the outer surface of the suspension liner; and
an adhesive that adheres at least the second and third portions of the insert to the suspension liner.

2. The system of claim 1 in which the first portion defines a projection that projects past the suspension liner's inner surface.

3. The system of claim 2 in which the projection is dome shaped.

4. The system of claim 1 in which the third portion has a larger surface area than does the first portion.

5. The system of claim 4 in which the third portion is a thin, flat structure, the second portion is a post that projects from the third portion, and the first portion is the distal end of the post.

6. The system of claim 5 in which the post is round and the third portion is generally rectangular.

7. The system of claim 4 in which the first, second and third portions are each thin, flat structures.

8. The system of claim 7 in which the insert comprises a conductive fabric.

9. The system of claim 8 in which the side of the fabric that is adjacent to the liner carries material that is essentially impervious to the adhesive.

10. The system of claim 9 in which the second portion is essentially fully wetted with material that is essentially impervious to air.

11. The system of claim 1 in which the insert is unitary.

12. The system of claim 11 in which the insert is made from a conductive elastomer.

13. The system of claim 12 in which the insert is molded from conductive elastomer.

14. The system of claim 1 further comprising a removable magnetic electrode that is magnetically coupled to the third portion of the insert, to allow varied positioning of the liner relative to the socket.

15. The system of claim 14 in which the magnetic electrode carries myoelectric signals from the insert to the socket.

16. A system for passing myoelectric signals through a suspension liner of a prosthetic device, the suspension liner having an inner surface that is in contact with a user's skin, and an outer surface that is adjacent to an inner or outer socket of the prosthetic device, the system comprising:
a multi-part electrode passing through the liner, the electrode comprising an inner portion located on the inner face of the liner and that presents a top that touches the user's skin, an outer portion located on the outer face of the liner, and an intermediate portion that electrically and mechanically interconnects the inner and outer portions,
wherein the faces of the inner and outer portions that are in contact with the liner are grooved, to increase the contact area between these faces and the liner, as well as to variably compress the liner material, both of which provide a tighter grip between the electrode and the liner.

17. The system of claim 16 in which the intermediate portion is a threaded stud that is received in a threaded bore in the outer portion.

18. The system of claim 16 further comprising a removable magnetic electrode that is magnetically coupled to the outer portion, to allow varied positioning of the liner relative to the socket.

19. The system of claim 16, further comprising a thin pickup electrode in the socket and in mechanical and electrical contact with the outer portion of the multi-part electrode, the thin pickup electrode comprising a thin inner disc that is thin enough to be easily conformed by the technician to the curvature of the socket.

20. A system for passing myoelectric signals through a suspension liner of a prosthetic device, the suspension liner having an inner surface that is in contact with a user's skin, and an outer surface that is adjacent to an inner or outer socket of the prosthetic device, the system comprising:
a flexible conductive fabric-based electrode insert defining a first portion located on or adjacent the inner surface of the suspension liner such that it touches the user's skin, a second portion passing through the suspension liner, and a third portion on the outer surface of the suspension liner, wherein all three portions are unitary, the first portion defines a projection that projects inwardly to slightly compress the user's skin, the third portion has a greater area than the first portion, and the second portion is essentially air-impervious; and
an adhesive that adheres the first, second and third portions of the insert to the suspension liner.

21. The system of claim 20 further comprising a film that is essentially impervious to the adhesive, located on the surface of the insert between the conductive fabric and the adhesive, to inhibit adhesive infiltration into the conductive fabric.
